(19) United States
(12) Plant Patent Application Publication
(10) Pub. No.: US 2025/0120328 P1
(43) Pub. Date: Apr. 10, 2025

(54) Apple Tree Named 'PremA047'

(50) ;

(71) Applicant: Prevar Limited, Hastings (NZ)

(72) Inventor: Richard Volz, Auckland (NZ)

(73) Assignee: Prevar Limited, Hastings (NZ)

(21) Appl. No.: 18/909,307

(22) Filed: Oct. 8, 2024

Related U.S. Application Data
(60) Provisional application No. 63/589,163, filed on Oct. 10, 2023.

(51) Int. Cl. A01H 6/74

(52) U.S. Cl. CPC A01H 6/7418
USPC PLT/161

(57) ABSTRACT

A new and distinct apple tree named ‘PremA047’, the fruit of which is notable for its intense red skin color, low susceptibility to russet, and excellent storage characteristics.

Latin name: Malus domestica.

Variety denomination: ‘PremA047’.

### BACKGROUND AND SUMMARY OF THE INVENTION

The new apple tree ‘PremA047’ was created in the course of a planned breeding program carried out at Havelock North, New Zealand. ‘PremA047’ originated as a result of a controlled cross made in 2009 of female parent ‘Scifresh’ (U.S. Plant Pat. No. 13,888) and male parent ‘A174R01T204’ (not patented). ‘PremA047’ was selected as a single plant from among a population of seedlings derived from the parents, and was initially observed for its attractive fruit with intense red skin color and low susceptibility to russet, its harvest timing, and for its excellent storage characteristics. The plant was first asexually propagated by grafting in 2014 at Havelock North, New Zealand, and has since been observed to retain its desirable characteristics and reproduce true to type over a number of asexually propagated generations.

‘PremA047’ is distinguished from parent variety ‘Scifresh’ and from similar variety ‘Galaxy’ (U.S. Plant Pat. No. 6,955) and ‘Fuji Supreme’ as set forth in Tables 1 and 2.

TABLE 1
Comparison of ‘PremA047’ to ‘Scifresh’

| Characteristic | ‘PremA047’ | ‘Scifresh’ |
|---|---|---|
| Fruit-skin | Yellow 4B to 4C | Yellow 8A |
| background color | | |
| Fruit-skin | Solid flush | Red 42C with |
| overcolor | greyed purple | narrow stripes |
| | 187A to 187B | of greyed-red |
| | over 90% to | 179A over |
| | 100% of skin | about 75% of |
| | surface | skin surface |

TABLE 2
Comparison of ‘PremA047’ ' to ‘Galaxy’

| Characteristic | ‘PremA047’ | ‘Galaxy’ |
|---|---|---|
| First bloom | Mid-September | 2 to 3 weeks after |
| | (Havelock North, NZ) | ‘PremA047’ |
| Pattern of | Solid flush | Striped |
| overcolor | | |

### BRIEF DESCRIPTION OF THE PHOTOGRAPHS

The attached photographs showing ‘PremA047’ apple trees and fruit were taken during the 2023 and 2024 growing season at Havelock North, New Zealand. The trees depicted were planted in 2016.

FIG. 1 shows fruit of ‘PremA047’;

FIG. 2 shows fruit of ‘PremA047’;

FIG. 3 shows a tree, fruit and leaves of ‘PremA047’;

FIG. 4 shows the upper surface of leaves of ‘PremA047’;

FIG. 5 shows the lower surface of leaves of ‘PremA047’;

FIG. 6 shows a tree, blossoms and young leaves of ‘PremA047’;

FIG. 7 shows blossoms of ‘PremA047’; and,

FIG. 8 shows flower buds of ‘PremA047’.

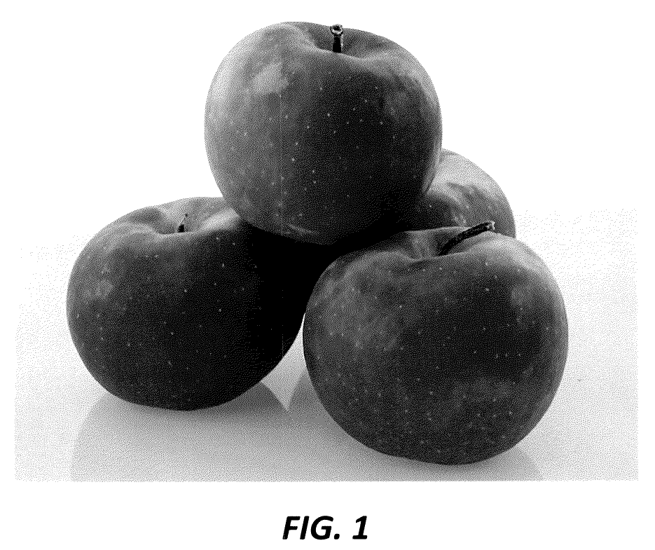

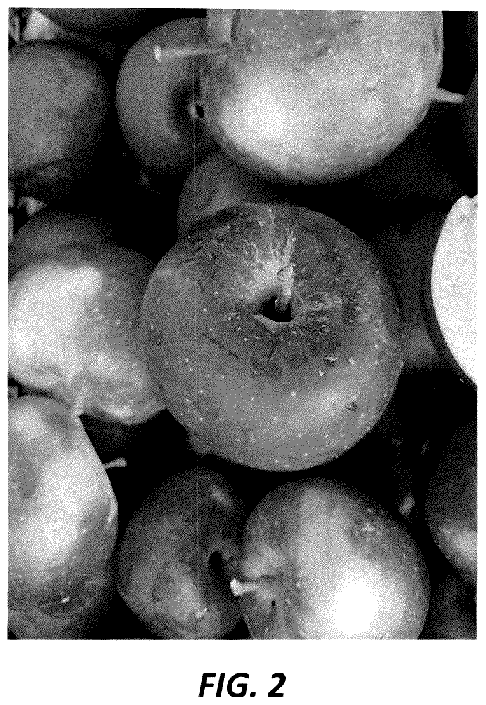

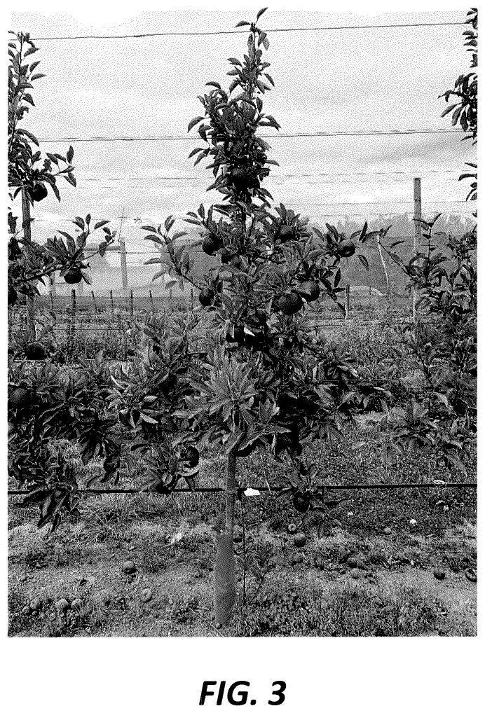

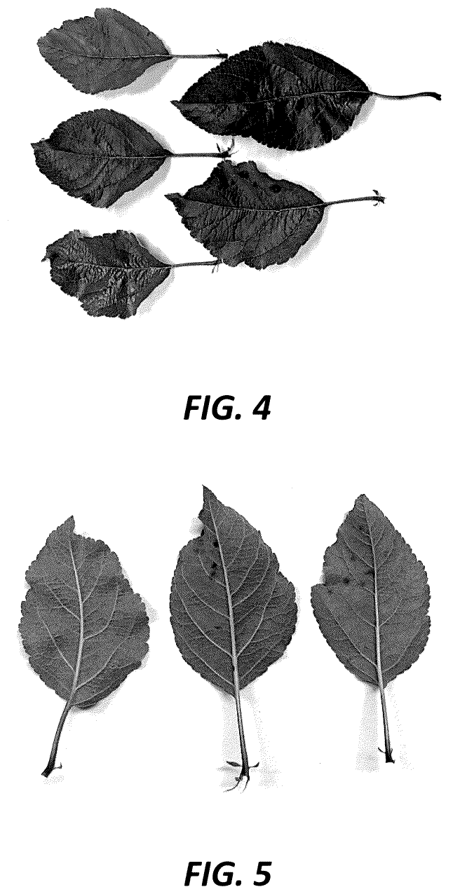

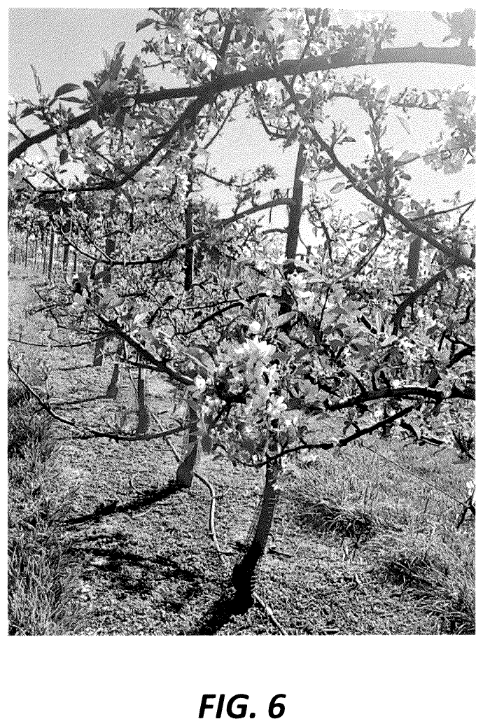

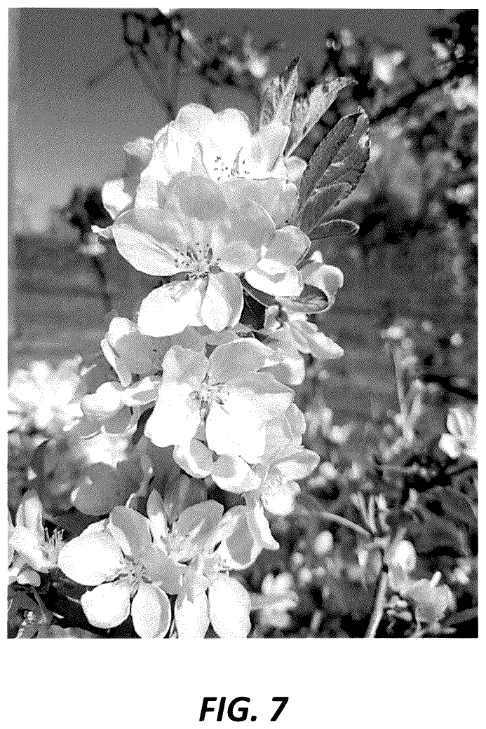

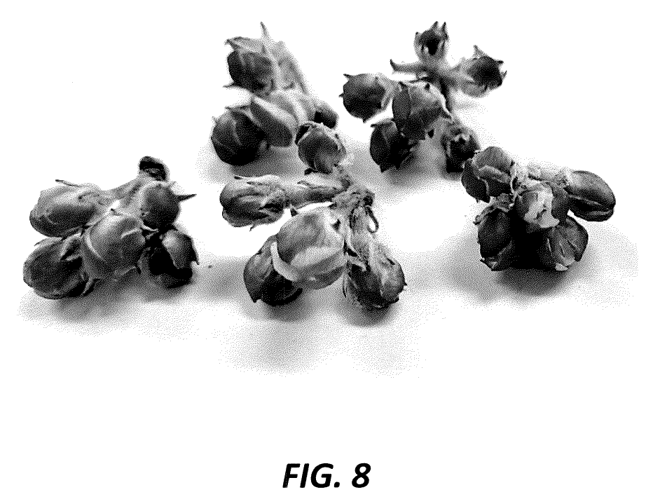

### BOTANICAL DESCRIPTION OF THE VARIETY

The following detailed botanical description is based on observations of ‘PremA047’ trees on ‘M9’ rootstock (not patented) planted at Havelock North, New Zealand in 2016. It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average. Color descriptions are made with reference to The R.H.S. Colour Chart, 6^{th} ed. (Royal Horticultural Society, London, 2015).
Tree: Trees of ‘PremA047’ are somewhat vigorous, have an upright to spreading growth habit, and tend to be shorter and more compact than ‘Galaxy’ Gala (U.S. Plant Pat. No. 6,955) and ‘CABp Fuji’ (U.S. Plant Pat. No. 17,914), while the trunk of ‘PremA047’ has a cross-sectional area similar to both varieties, as detailed in Table 3.

TABLE 3
Comparison of height and trunk
cross-sectional area at third leaf

| | Tree | Trunk Cross- | |
|---|---|---|---|
| | Height | Sectional Area | |
| PremA047 | 2.6 m | 10 | cm² |
| Galaxy Gala | 3.3 m | 10.1 | cm² |
| CABp Fuji | 3.0 m | 10 | cm² |

Vigor.—Medium.
Type.—Ramified.
Habit.—Upright to spreading.
Spread of mature tree.2.1 m.
Height.3.4 m.
Trunk diameter at 30 cm above graft.19.9 cm.
Bearing.—On spurs.
Bark texture.—Medium.
Bark color.—Grey-brown 197C.
Lenticel length.6 mm.
Lenticel width.2 mm to 3 mm.
Lenticel shape.—Elongated.
Lenticel color.—Grey-brown N199A.
Lenticel density.16 to 22 per 10 cm².
Fruiting branches:
   Length.77.2 cm.
   Diameter.13.5 mm.
   Crotch angle.80° to 95° (trained).
   Bark color.—Grey-brown N199C.
   Lenticel length.1.9 mm to 3.1 mm.
   Lenticel width.1 mm to 1.2 mm.
   Lenticel shape.—Elongated and vertically oriented.
   Lenticel color.—Yellow-green153B.
   Lenticel density.5 to 7 per cm².
One-year-old shoot:
   Length.29.2 cm.
   Diameter.7.7 mm.
   Pubescence.—Present.
   Color.—Grey-brown N199D.
   Lenticel length.1.5 mm to 2 mm.
   Lenticel width.0.5 mm to 1 mm.
   Lenticel shape.—Round to elongated.
   Lenticel density.6 per cm².
Flowers: ‘PremA047’ blooms 2 to 3 weeks earlier than ‘Galaxy’, with a bloom period of 3 to 4 weeks. The presence of the S₂ and S₅ self-incompatibility alleles in ‘PremA047’ makes it pollen-compatible with nearly all major commercial cultivars grown in the area where tested.
Blower buds:
   State described.—First pink.
   Quantity per spur.5.1 (mean).
   Bud shape.—Round.
   Apex shape.—Rounded.
   Length.9.4 mm.
   Diameter.8.2 mm.
   Color.—Red-purple 64B at first pink; Purple 76D at balloon stage.
Blooms:
   Diameter of fully open flower.37 mm.
   Depth of fully open flower.12.5 mm.
   Relative position of petal margin.—Not touching.
   Average number of flowers per cluster.4.4.
   First bloom.—Sep. 21, 2023 (Havelock North, NZ).
   Full bloom.—Oct. 7, 2023 (Havelock North, NZ).
   Pollinator.—Malus floribunda.
Flower Petals:
   Number per flower.5.
   Petal shape.—Obovate.
   Length.18 mm.
   Width.11.6 mm.
   Apex shape.—Rounded to obtuse.
   Base shape.—Obtuse.
   Margin.—Mildly undulating.
   Color—upper surface.—White N155B.
   Color—lower surface.—Purple 76D with streaks of purple 76B.
Pistils:
   Length.8.4 mm.
   Color.—Yellow-green 145C.
Stigma:
   Diameter.0.5 mm to 1 mm.
   Color.—Yellow-green 151C.
Style:
   Length.8.9 mm.
   Color.—Yellow-green 151C.
Stamens:
   Quantity per flower.14.8 mm.
   Length.9 mm.
   Anther color.—Yellow 3B.
   Pollen.—Present, abundant.
Pedicel:
   Length.23 mm.
   Diameter.1.5 mm.
   Color.—Yello-green 144B with greyed-orange 177A.
Sepals:
   Quantity per flower.5.
   Color.—Yellow-green 145A.
   Shape.—Broad triangle.
   Apex shape.—Acute.
   Margin.—Smooth, reflexed.
Leaves:

Shape Ovate
Length.103 mm.
Width.56 mm.
Length to width ratio.1.84.
Margin.—Crenate.
Apex shape.—Acuminate.
Base shape.—Oblique.
Color—upper surface.—Green N137B.
Color—lower surface.—Yellow-green 144A.
Attitude in relation to shoot.—Upwards.
Petiole:
   Length.32.6 mm.
   Diameter.1.9 mm.
   Color.—Yellow-green 145B; greyed-purple 184B anthocyanin present primarily at attachment end.
Fruit: The mean fruit weight for ‘PremA047’ fruits from 3rd leaf trees is 199 g, which is similar to fruits of ‘CABp Fuji’ and smaller than fruits of ‘Galaxy’ gala. The shape of fruits of ‘PremA047’ is globose-conical and symmetrical, with the broadest point at the fruit equator or above. Harvest yield for third leaf trees of ‘PremA047’ ranged from 14.5 to 20.3 metric tons per hectare, with an average of 18 MT/ha. Fruits of ‘PremA047’ have been observed to have a pleasing fruity flavor with low to moderate acid-sugar balance. When comparing the harvest characteristics of ‘PremA047’ fruit with those of ‘Galaxy’ Gala and ‘CABp Fuji’, ‘PremA047’ fruit was observed to be 0.7 to 0.8 kgf more firm than ‘Galaxy’ Gala and 1.2 to 1.4 kgf more firm than ‘CABp Fuji’. ‘PremA047’ fruit had similar soluble solids and acidity contents to ‘CABp Fuji’ and had 1.8% to 2.0% higher soluble solids content as compared to ‘Galaxy’.
   Quantity per fluster.2 to 3 after thinning.
   Height.65.7 mm.
   Diameter.83.6 mm.
   Ratio of height to width.0.79.
   Weight.235.7 g.
   General shape in profile.—Globose conical.
   Position of maximum diameter.—At or above equator.
   Ribbing.—Moderate.
   Crowning at calyx end.—Absent to weak.
   Size of eye.—Medium.
   Aperture of eye.—Closed.
   Depth of eye basin.33.12 mm.
   Width of eye basin.10.12 mm.
   Length of sepal.4 mm to 7 mm.
   Bloom of skin.—Present in moderate amount.
   Greasiness of skin.—Weak.
   Background color of skin.—Yellow 4B to 4C.
   Over color of skin.—Grey-purple 187A to 187B.
   Amount of over color.—High to very high, 90% to 100%.
   Intensity of over color.—Very dark.
   Pattern of over color.—Solid flush.
   Amount of russet around stalk cavity.—Absent to small.
   Amount of russet on cheeks.—Minimal.
   Area of russet around eye basin.—Small.
   Length of stalk.22.17 mm.
   Thickness of stalk.2.66 mm.
   Depth of stalk cavity.17.68 mm.
   Width of stalk cavity38 64 mm.
   Stalk color.—Greyed-orange 166B.
   Firmness of flesh.8.66 kgf.
   Flesh texture.—Fine to medium.
   Aroma.—Absent to very weak.
   Juiciness.—Medium.
   Brix.15.7%.
   Flesh color.—Yellow 8D.
   Number of lenticels.—Few to medium.
   Size of lenticels.—Medium.
Seeds:
   Quantity per fruit.5 to 8.
   Shape.—Ovate.
   Color.—Brown 200B.
   Length.9.57 mm.
   Width.5.83 mm.
Harvest:
   Date.—Apr. 2, 2024 (after ‘Scifresh’, similar to ‘Scilate’ U.S. Plant Pat. No. 20,477).
   Number of picks.—One.
   Number of fruits produced per tree per harvest60+.
Disease and pest resistance and susceptibility: ‘PremA047’ is susceptible to black spot, fire blight, and is sensitive to European canker.
Storageability: Stores well for 6 months with 1-MCP; exhibits a very low incidence of fruit storage disorders, with incidence less than 1% for soft scald and 1.5% for lenticel defect.
Market use: Fresh local market and export

1. A new and distinct apple tree named ‘PremA047’ substantially as described and illustrated herein.
